(19) United States
(12) Patent Application Publication
(10) Pub. No.: US 2015/0367025 A1
(43) Pub. Date: Dec. 24, 2015

(54) MEDICAL PRODUCT

(71) Applicant: AESCULAP AG, Tuttlingen (DE)

(72) Inventors: Christof Merckle, Mannheim (DE); Erich Odermatt, Schaffhausen (CH)

(21) Appl. No.: 14/765,588

(22) PCT Filed: Mar. 5, 2014
(86) PCT No.: PCT/EP2014/054213

§ 371 Date: Aug. 4, 2015

(30) Foreign Application Priority Data
Mar. 8, 2013 (DE) 10 2013 203 997.2

(51) Int. Cl. A61L 26/00

(52) U.S. Cl. CPC A61L 26/0033; A61L 26/0076; A61L 26/0019; A61L 26/009; A61L 2400/04

(57) ABSTRACT

A medical product is in the form of a particulate and/or fibrous flock material or of a suspension or paste containing a particulate and/or fibrous flock material. A discharge device contains the medical product. A medical kit includes the medical product and at least one further component selected from the group including a suture material, a stapler, a discharge device, more particularly a spray device, for the medical product and combinations thereof.

### TECHNICAL FIELD

This disclosure relates to a medical product and the application thereof in the area of medicine.

### BACKGROUND

Medical products such as, for example, spongy or foamy wound coverings are described in EP 2 233 157 A1.

An occasional problem in known medical products is their application into poorly accessible body defect zones.

Moreover, there is a certain risk that—depending on the material used—contracting of the material and thus an inhomogeneous distribution of the material in a body defect zone can occur, thereby endangering the effect intended from a medical point of view.

A further problem in known products is that they do not always optimally promote wound healing.

### SUMMARY

We provide a medical product in the form of a particulate and/or fibrous flock material or of a suspension or paste containing a particulate and/or fibrous flock material.

We also provide a discharge device containing the medical product in the form of a particulate and/or fibrous flock material or of a suspension or paste containing a particulate and/or fibrous flock material.

We further provide a medical kit including the medical product in the form of a particulate and/or fibrous flock material or of a suspension or paste containing a particulate and/or fibrous flock material and at least one further component selected from the group consisting of a suture material, a stapler, a discharge device, a spray device for the medical product and combinations thereof.

### DETAILED DESCRIPTION

We provide a medical product in the form of a particulate and/or fibrous flock material.

Alternatively, the medical product can be in the form of a solution, dispersion, suspension or paste which contains the particulate and/or fibrous flock material or is produced using the particulate and/or fibrous flock material.

We surprisingly found that particulate and/or fibrous flock material as such is usable as a medical product and/or to produce medical solutions, dispersions, suspensions or pastes.

The medical product has the following advantages:

Owing to the particulate and/or fibrous design of the flock material, the medical product has an enlarged surface area, and this is an advantage especially in wound coverings, in stopping bleeding and/or wound healing. Wound healing can be additionally improved by additization with wound healing-promoting substances.

After application of the medical product to or into a body defect zone, the particulate and/or fibrous flock material can, with particular advantage, form a kind of “three-dimensional structure” or “matrix” having cavities or pores (between the applied flock particles and/or fibers), favoring ingrowth of cells, extracellular matrix or tissue and thus the wound healing process.

A further advantage associated with the enlarged surface area of the flock material is that generally relatively low amounts of the medical product are sufficient to achieve the effect desired from a medical point of view.

A further advantage of the medical product arising from the particulate and/or fibrous design of the flock material is that the product can be distributed more uniformly or more homogeneously on or in a body defect zone, ensuring that sufficient material to achieve the intended medical effect is available on or in the body defect zone.

Any visible inhomogeneities in the distribution of the medical product can be easily compensated for by “re-application” of the product before or during a treatment step.

A further advantage is that, owing to the particulate and/or fibrous design of the flock material, the medical product allows complete coverage of a body defect zone to be treated, for example, a wound without complicated adaptations of the product to the proportions of the body defect zone to be treated being necessary before or during a medical procedure, more particularly a surgical procedure.

Another advantage is that, owing to the particulate and/or fibrous design of the flock material, the medical product can be applied in a targeted manner to or into a body defect zone.

Owing to the particulate and/or fibrous design of the flock material and the associated surface area enlargement, it is additionally possible to more easily and, in particular, more rapidly apply the medical product to or into a body defect zone. Depending on the form of the product, the latter can be applied as, for example, a dispersion or suspension by spraying (or optionally by spreading) or a paste by brushing to or into the body defect zone. This likewise contributes to facilitating the desired treatment.

As a result, the medical product can also be applied without relatively great difficulties to or into poorly accessible body defect zones.

A further advantage is that, owing to the particulate and/or fibrous design of the flock material, the medical product can be applied to or into a body defect zone in reproducible and, in particular, definable amounts, making it possible to carry out a medical procedure, more particularly a surgical procedure, under similarly reproducible and, in particular, definable conditions.

Through the specific selection of materials and/or additives for the flock material, it is additionally possible to specifically influence or adjust the properties thereof such as, for example, absorbency, swellability, adhesiveness and resorbability. This allows indication-specific adaptation of the product.

Lastly, one advantage is that the flock material and thus the product can be produced in a cost-effective manner. For instance, the flock material can, for example, be extruded and transferred to a particulate and/or fibrous form.

The term “fibrous flock material” or “flock fibers” means fibers having a defined fiber length (preferred fiber lengths will be mentioned below). The fibrous flock material is usually produced by trimming to length, more particularly cutting, or milling of single fibers (for example, extrusion fibers), more particularly so-called continuous fibers.

The medical product is preferably a wound covering agent, more particularly a surgical wound covering agent. The medical product is present especially in the form of a spray bandage. The medical product is preferably suitable for use in the treatment of surgical wounds and/or for the treatment of external wounds such as, for example, burn wounds and/or chronic wounds and/or for the treatment of internal wounds such as, for example, parenchymatous wounds. A further possible area of application concerns the use of the medical product for use in the treatment of ulcers.

The medical product may be a haemostatic (styptic), i.e., an agent that stops bleeding. The medical product can be intended especially for use in the treatment of diffuse bleeding and/or heavy bleeding such as, for example, parenchymatous bleeding.

The medical product may be a filling material, especially for filling in or augmenting soft tissue defects such as, for example, cartilage defects and/or hard tissue defects such as, for example, bone defects.

The medical product may be an occlusion agent. The medical product may also be used for closing, occluding or sealing dura mater defects. The medical product may be used for sealing or occluding air and/or liquid leaks in the human and/or animal body. The medical product may be used for sealing or occluding puncture channel bleeding.

The medical product may be used for use in negative-pressure therapy or negative-pressure sealing.

Preferably, the fibrous flock material is flock fibers, preferably monofilament flock fibers.

Particularly preferably, the fibrous flock material is present in the form of single flock fibers. In other words, it is particularly preferred when the fibrous flock material is not connected, especially not via an adhesive layer, to a substrate, more particularly an implant body and/or a supporting element, or assembled to form a planar structure such as, for example, a non-woven fabric, non-woven scrim, loop-drawingly knitted fabric, loop-formingly knitted fabric or the like.

The fibrous flock material can, in principle, have a fiber length of 40 μm to 15 mm, more particularly 50 μm to 10 mm, preferably 100 μm to 5 mm. The fibrous flock material can, in particular, have a fiber length of 10 μm to 5 mm, more particularly 40 μm to 4 mm, preferably 50 μm to 3 mm, more preferably 100 μm to 2 mm. The fibrous flock material can, with particular preference, have a fiber length of 100 μm to 3 mm, preferably 200 μm to 2 mm.

Furthermore, the fibrous flock material can have a fiber thickness or fiber diameter of 20 μm to 900 μm, more particularly 70 μm to 600 μm, preferably 100 μm to 400 μm.

In addition, the fibrous flock material can have a linear density of 0.01 dtex to 1000 dtex, more particularly 0.1 dtex to 500 dtex, preferably 0.3 dtex to 200 dtex. The fibrous flock material can, in particular, have a linear density of 0.01 dtex to 100 dtex, preferably 0.1 dtex to 80 dtex, more preferably 0.3 dtex to 40 dtex. The dimension “dtex” (decitex) means a linear density of 1 g per 10 000 m length of the fibrous flock material.

The fibrous flock material can be textured or non-textured. Furthermore, the fibrous flock material can have a circular cross section or a non-circular cross section, more particularly an oval, ellipsoidal, polygonal, for example, triangular, rectangular, square, rhomboidal, pentagonal, hexagonal and/or star-shaped, cross section.

The particulate flock material can be in the form of a powder or granules. Preferably, the particulate flock material is milled fibers, more particularly milled flock fibers. The particulate flock material can, in particular, have a particle size or particle diameter of 0.05 μm to 5 mm, more particularly 0.1 μm to 3 mm, preferably 10 μm to 2 mm.

The flock material may comprise a mucoadhesive material or be formed from such a material. More particularly, the flock material can comprise a material or be formed from a material capable of binding to biological tissue, more particularly human and/or animal tissue, via ionic interactions and/or the formation of hydrogen bonds. Suitable materials may comprise functional groups preferably selected from the group comprising carboxylate groups, phosphate groups, sulphate groups, sulphonate groups, amino groups and combinations thereof. The examples described in this paragraph are especially advantageous with regard to wound-covering and/or haemostatic uses of the product.

To increase the dimensional stability of the product, it can additionally be provided that the flock material comprises a crosslinked material or is formed from such a material. A dimensionally stable product is especially advantageous with regard to use as filling material. In this case, the material can be physically and/or chemically crosslinked. It is possible to achieve physical crosslinking of the material by, for example, irradiation techniques suitable for this purpose. Corresponding techniques are familiar to those skilled in the art, and so further explanations will not be given here. By contrast, chemical crosslinking of the material is generally based on the use of a suitable crosslinker. For example, the flock material can be crosslinked with a chemical crosslinker selected from the group comprising aldehydes such as, for example, formaldehyde, dialdehydes such as, for example, glutaraldehyde, polyaldehydes such as, for example, dextran aldehyde, carbodiimides, diisocyanates such as, for example, hexamethylene diisocyanate, salts thereof and mixtures thereof.

Preferably, the flock material comprises a resorbable material or is formed from such a material. The surface area enlargement associated with the particulate and/or fibrous design of the flock material may promote rapid resorption of the medical product.

As an alternative or in addition to the preceding example, the flock material can comprise a non-resorbable material or be formed from such a material. From a dimensional stability point of view and from a load-bearing point of view, a non-resorbable flock material can be especially advantageous when using the product as a filling material.

The material described for the flock material can be of synthetic, biological and/or recombinant origin. Furthermore, the material for the flock material can be of natural or native origin. The material can, in particular, be of xenogeneic origin, preferably porcine, bovine and/or equine origin. Alternatively, the material for the flock material can also be of human origin. It is additionally preferred when the flock material is present in particulate form.

Preferably, the material for the flock material is a polymer, selected in particular from the group comprising synthetic polymer, recombinant polymer, naturally occurring polymer or biopolymer and mixtures, more particularly blends, thereof. More particularly, the polymer can be a copolymer. The expression “copolymer” means a polymer composed of at least two different monomer units. Thus, “copolymer” encompasses not only copolymers in the narrower sense, i.e., so-called bipolymers (polymers composed of two different monomer units), but also terpolymers, tetrapolymers and the like. The copolymer can additionally be selected from the group comprising random copolymer, alternating copolymer, block copolymer or segmented copolymer, graft copolymer and mixtures, more particularly blends, thereof.

Preferably, the flock material comprises a resorbable material or is formed from such a material selected from the group comprising polyhydroxyalkanoates, proteins, extracellular proteins, serum proteins, polysaccharides, mucopolysaccharides, carboxyl group-bearing polysaccharides, amino group-bearing polysaccharides, aldehyde group-bearing polysaccharides, copolymers thereof, salts thereof, stereoisomers, more particularly diastereomers, thereof and mixtures, more particularly blends, thereof.

More particularly, the flock material can comprise a resorbable material or be formed from such a material selected from the group comprising polyglycolide or polyglycolic acid, polylactide or polylactic acid, polydioxanone, poly-3-hydroxybutyrate or poly-3-hydroxybutyric acid, poly-4-hydroxybutyrate or poly-4-hydroxybutyric acid, polytrimethylene carbonate, poly-ε-caprolactone, polyvinyl alcohol, cotton, cellulose, cellulose derivatives such as, for example, alkylcelluloses, methylcellulose, hydroxyalkylcelluloses, hydroxymethylcellulose, hydroxyethylcellulose, hydroxypropylcellulose, carboxyalkylcelluloses, carboxymethylcellulose, starch, amylose, amylopectin, dextran, dextrin, chitin, chitosan, hyaluronic acid, dextran sulphate, heparin, heparan sulphate, chondroitin sulphate, dermatan sulphate, collagen, gelatin, elastin, reticulin, fibronectin, laminin, fibrin, fibrinogen, albumin, copolymers thereof, salts thereof, stereoisomers, more particularly diastereomers, thereof and mixtures, more particularly blends, thereof.

As an alternative or in addition, the flock material can comprise a non-resorbable material or be formed from such a material which is selected from the group comprising polyolefins, polyamides, polyesters, polyurethanes, more particularly thermoplastic polyurethanes, copolymers thereof and mixtures, more particularly blends, thereof.

More particularly, the flock material can comprise a non-resorbable material or be formed from such a material which is selected from the group comprising polyethylene, low-density polyethylene, high-density polyethylene, high-molecular-weight polyethylene, ultra-high-molecular-weight polyethylene, polypropylene, polyethylene terephthalate, polypropylene terephthalate, polybutylene terephthalate, polyacrylonitrile, nylon-6, nylon-6,6, nylon-6,12, nylon-12, silk, more particularly rayon or spider silk, polytetrafluoroethylene, more particularly expanded polytetrafluoroethylene (ePTFE), polyvinylidene difluoride, polytetrafluoropropylene, polyhexafluoropropylene, copolymers thereof and mixtures, more particularly blends, thereof.

The flock material can comprise a material having a melting point below 100° C., in particular having a melting point from 70° C. to 100° C., or can be formed of such a material.

Advantageously, the flock material can be additized, i.e., comprise one or optionally more additives.

To achieve detectability, especially by imaging methods such as, for example, MRI methods (Magnetic Resonance Imaging methods), it can be provided that the flock material comprises a radiopaque additive such as, for example, barium sulphate, especially in the form of particles, or a metal or nitinol thread.

To achieve a biological, pharmaceutical and/or medical effect, it can be provided alternatively or additionally that the flock material comprises an active ingredient selected from the group comprising biological active ingredient, pharmaceutical active ingredient, medical active ingredient and mixtures thereof.

More particularly, the flock material can comprise an active ingredient selected from the group comprising antimicrobial, more particularly antibiotic, active ingredient, wound healing-promoting active ingredient, disinfecting active ingredient, anti-inflammatory active ingredient, blood coagulation-promoting active ingredient, growth factors, cell-differentiating factors, cell-adhesive factors, cell-recruiting factors, cell receptors, cell-binding factors, cytokines, peptides, structural proteins, extracellular proteins such as, for example, collagen, serum proteins such as, for example, albumin, polysaccharides such as, for example, hyaluronic acid, oligonucleotides, polynucleotides, DNA, RNA, salts thereof, stereoisomers, more particularly diastereomers, thereof and mixtures thereof.

For example, the flock material can comprise an active ingredient selected from the group comprising biguanides, polyhexamethylene biguanide (PHMB), triclosan, chlorhexidine, gentamicin, vitamins, copper, zinc, silver, gold, salts thereof, stereoisomers, more particularly diastereomers, thereof and mixtures thereof.

We further provide that the medical product is in the form of a mixture of different particulate and/or fibrous flock materials. In this case, the flock materials can differ from one another with regard to at least one parameter preferably selected from the group comprising particle size (in the case of a particulate flock material), fiber length (in the case of a fibrous flock material), thickness or diameter, linear density (in a fibrous flock material), material and mixtures thereof. With regard to the parameters listed in this paragraph, full reference is made to the description so far.

Advantageously, the medical product is sprayable, more particularly aerosolizable. The solution, dispersion, suspension or paste referred to above in connection with the product additionally comprises a biocompatible solution agent, dispersion agent or suspension agent, generally water or an aqueous mixture, and/or a biocompatible carrier substance such as, for example, fats, oils, glycerol or the like.

If the product is present in the form of a suspension, it can be provided that the suspension has a flock material content of 1% by weight to 20% by weight, more particularly 3% by weight to 15% by weight, preferably 5% by weight to 10% by weight, based on the total weight of the suspension.

The medical product is present in sterile and, in particular, off-the-shelf form. Sterilization of the product can, for example, be achieved using ethylene oxide and/or gamma irradiation.

We provide a discharge device containing the medical product described above.

The discharge device is preferably designed as a spray device.

The discharge device can, in particular, be used for discharging an aerosol. In this case, it may be useful for the discharge device to contain a suitable propellant in addition to the medical product. A suitable propellant can be selected from the group comprising perfluorinated ethers (for example, Desfluran®), fluorinated hydrocarbons, more particularly fluorinated hydrocarbons of medium chain length (chain length having from two to five carbon atoms) such as, for example, tetrafluoroethane, hexafluoropropane, heptafluoropropane, decafluorobutane, octafluorocyclobutane, noble gases, nitrogen, oxygen, carbon dioxide, dinitrogen oxide and mixtures thereof.

With regard to further features and advantages of the discharge device, especially the medical product and/or a possible propellant, full reference is made to the description above.

We also provide a medical kit comprising the medical product described above or the discharge device described above and also at least one further component preferably selected from the group comprising a suture material, a stapler, a discharge device, more particularly a spray device, for the medical product and combinations thereof.

With regard to further features and advantages of the kit, in particular the medical product and/or the discharge device, full reference is made to the description above.

Further features and advantages are revealed by the following description of preferred examples. Individual features can, in each case, be realized on their own or in combination with one another. The preferred examples serve solely to further elucidate the disclosure and to provide a better understanding, without restricting the disclosure thereto.

### EXAMPLES

### 1. Production of Flock Fibers Composed of Poly(ε-caprolactone-co-trimethylene carbonate)

An extrusion device was used to extrude fibers composed of poly(ε-caprolactone-co-trimethylene carbonate). The fibers were subsequently trimmed to flock fibers of 100 μm in length using a precision cutting device. The produced flock fibers composed of poly(ε-caprolactone-co-trimethylene carbonate) were subsequently sterilized using ethylene oxide and lastly dried.

It was possible to use the flock fibers successfully in the coverage of wounds and as a haemostatic.

### 2. Production of Particulate Flock Material Composed of Collagen

Starting from bovine skin, collagen-containing granules were produced and subsequently finely ground. This gave rise to a collagen-containing flock material having a particle size of 10 μm to 80 μm, depending on the milling operation.

It was similarly possible to use the collagen-containing flock material successfully for wound coverage and stopping bleeding.

### 3. Treatment of a Superficial Wound

Using a scalpel, a 5 cm long incision was made on the right shank of a pig.

The resulting wound was cleaned. Thereafter, collagen-containing flock fibers of 100 μm in length were sprayed onto the wound until the wound was completely covered. The flock fibers fixed in the wound by themselves. It was possible to easily remove excess flock fibers outside the wound.

The covered wound was subsequently further treated as part of a negative-pressure therapy, with the “porous” structure formed by the sprayed-on flock fibers allowing cell transfer and tissue transfer into the wound region and drying of the wound. At the same time as new skin formation, the flock fibers were resorbed, finalizing wound closure with endogenous skin.

1-18. (canceled)

19. A medical product in the form of a particulate and/or fibrous flock material or of a suspension or paste containing a particulate and/or fibrous flock material.

20. The medical product according to claim 19, wherein the product is a wound covering agent in the form of a spray bandage.

21. The medical product according to claim 19, which is a haemostatic.

22. The medical product according to claim 19, wherein the fibrous flock material is in the form of monofilamemt flock fibers.

23. The medical product according to claim 19, wherein the fibrous flock material has a fiber length of 10 μm to 5 mm.

24. The medical product according to claim 19, wherein the fibrous flock material has a linear density of 0.01 dtex to 100 dtex.

25. The medical product according to claim 19, wherein the particulate flock material has a particle size or particle diameter of 0.05 μm to 5 mm.

26. The medical product according to claim 19, wherein the flock material comprises a mucoadhesive material or is formed from such a material.

27. The medical product according to claim 19, wherein the flock material comprises a material or is formed from a material capable of binding to biological tissue via ionic interactions and/or formation of hydrogen bonds.

28. The medical product according to claim 19, wherein the flock material comprises a material or is formed from a material which comprises functional groups selected from the group consisting of carboxylate groups, sulphate groups, sulphonate groups, phosphate groups, amino groups and combinations thereof.

29. The medical product according to claim 19, wherein the flock material comprises a crosslinked material or is formed from such a material.

30. The medical product according to claim 19, wherein the flock material comprises a resorbable material or is formed from such a material.

31. The medical product according to claim 19, wherein the flock material comprises a resorbable material or is formed from a resorbable material selected from the group consisting of polyhydroxyalkanoates, proteins, extracellular proteins, serum proteins, polysaccharides, mucopolysaccharides, carboxyl group-bearing polysaccharides, amino group-bearing polysaccharides, aldehyde group-bearing polysaccharides, copolymers thereof, salts thereof, stereoisomers, diastereomers thereof, mixtures and blends thereof.

32. The medical product according to claim 19, wherein the flock material comprises a non-resorbable material or is formed from such a material.

33. The medical product according to claim 19, wherein the flock material comprises a non-resorbable material or is formed from a non-resorbable material which is selected from the group consisting of polyolefins, polyamides, polyesters, polyurethanes, more particularly thermoplastic polyurethanes, copolymers thereof, mixtures and blends thereof.

34. The medical product according to claim 19, which is sprayable or aerosolizable.

35. A discharge device containing the medical product according to claim 19.

36. A medical kit comprising the medical product according to claim 19 and at least one further component selected from the group consisting of a suture material, a stapler, a discharge device, a spray device for the medical product and combinations thereof.
